(19) United States
(12) Patent Application Publication
(10) Pub. No.: US 2004/0064125 A1
(43) Pub. Date: Apr. 1, 2004

(54) Pleated tissue and adhesive arrangement for the absorbent core of an extensible absorbent article

(76) Inventors: David A. Justmann, Hortonville, WI (US); Barbara A. Gossen, Oshkosh, WI (US); Thomas W. Van Eperen, Kaukauna, WI (US)

Correspondence Address: PAULEY PETERSEN KINNE & ERICKSON, 2800 WEST HIGGINS ROAD, SUITE 365, HOFFMAN ESTATES, IL, 60195, US

(21) Appl. No.: 10/260,103

(22) Filed: Sep. 30, 2002

(51) Int. Cl. A61F 13/15; A61F 13/20

(52) U.S. Cl. 604/385.23

(57) ABSTRACT

An extensible absorbent article or garment has an extensible top sheet layer for facing the body of the wearer; an extensible back sheet layer for an exterior layer facing away from the body of the wearer; and an absorbent core layer located between the top sheet and the back sheet. The absorbent core has a tissue layer acting as a boundary layer or web between the top sheet and the fluff pad of the absorbent core. The tissue web of the absorbent core has an adhesive discontinuously applied thereto for attachment to the top sheet or other extensible layers to avoid tearing the tissue when the extensible layers expand. The tissue web may further be pleated to allow for its extensibility. The discontinuity of the adhesive then allows the pleats to perform their expansion function unbound and thus the tissue can extend without tearing, thereby maintaining the integrity of the tissue barrier.

### BACKGROUND OF THE INVENTION

Personal care products including incontinence garments such as limited use or disposable diapers and adult incontinence garments; and sanitary pads often are made with a top sheet material (also referred to as a cover sheet or liner), an absorbent core, and a liquid impervious back sheet, or outer layer. Some such items may also have a surge layer for fluid uptake and distribution, or other specialized layers between the top sheet and absorbent core. Absorption and retention of fluid, comfort, and avoidance of leakage are the functions desired of such products.

Typically, the absorbent core is the primary liquid retention layer and includes a web of fibers loosely holding an amount of superabsorbent particles. The web of fibers provides structure for the absorbent core and optionally contributes to absorbency. Superabsorbent particles are added to the web to increase the absorbency and retention effectiveness of the webs on a unit mass basis. In order to contain the particulate superabsorbent in place at the absorbent core, the absorbent core is surrounded by a barrier layer of tissue. If not contained, the particulate superabsorbent might otherwise travel out of the absorbent core and through the top sheet to contact the wearer. In such cases, when the particulate superabsorbent is wetted it may gel on the skin of the wearer. Thus, uncontained superabsorbent can present aesthetic problems, functional degradation of the garment, or both.

There has been a desire in the art to make incontinence garments, such as diapers, better fitting. One technique for rendering a better fit is to have the top and back sheets extensible, especially laterally or transversely, and especially, although not necessarily exclusively, in the waist areas of the garment. It is known in the art that extensibility of the garment is limited by the least extensible layer when said layers are connected in the constructed garment.

In order to keep the various functional layers in place in the constructed garment, the absorbent core is typically adhered to one of the extensible top or back sheets. This presents a problem for extensible garments in that the tissue barrier layer covering the superabsorbent particles (SAP) loaded web has little or no expandibility. Because the tissue layer is adhered to, e.g., the liner layer, when the liner layer expands, as it will when the diaper is loaded with exudates, or is pulled on over the hips of the wearer, or stretched to fit and fasten on the wearer, the tissue layer may tear. If the tissue layer tears, it no longer acts as a barrier to the fine grain superabsorbent particles contained within the absorbent core, resulting in the problems discussed above.

Therefore, there exists a need or desire for an improved integrity of the barrier layer of the absorbent structure, in order that the absorbent materials of the absorbent core remain in place.

### SUMMARY OF THE INVENTION

The present invention provides solutions to the above-described need in its various aspects. By way of general summary, the present invention may provide an extensible absorbent article having an extensible liner layer for facing the body of the wearer; an extensible back sheet layer for an exterior layer facing away from the body of the wearer; and an absorbent core layer located between the liner and the back sheet. The extensible liner layer may include the so-called “surge” management layer which facilitates rapid uptake and distribution of liquids. In such case the surge management layer will also be extensible. The absorbent core will generally have a tissue layer acting as a boundary, or barrier layer, between the liner, and surge management material if present, and the absorbent materials of the absorbent core. In order to enhance the utility of the tissue barrier, aspects of the present invention provide that the tissue barrier be adhered to at least one of the extensible layers of the article in such a manner as to allow the extensible layers to expand without damaging the tissue barrier by placement of specifically designated patterns of adhesive. Some adhesive patterns may also be used to create dimensional stability in areas where the extensible layers of the garment are not desired to be extensible.

The absorbent core may have a folded, i.e. pleated, tissue layer acting as a boundary, or barrier layer, between the liner and the absorbent materials of the absorbent core. In order to enhance the utility of the pleated tissue barrier, aspects of the present invention provide that the tissue barrier be adhered to at least one of the naturally extensible layers of the article with specifically designated patterns of applied adhesive.

In certain aspects of the present invention, the tissue web of the absorbent core can be pleated to allow for its extendibility and have an adhesive discontinuously applied thereto for attachment to the extensible layers of the garment. The discontinuity of the adhesive allows the pleats to perform their expansion function unbound and thus the tissue can expand along with the liner without tearing, thereby maintaining the integrity of the tissue barrier.

The adhesive discontinuity can be done in a variety of manners taught herein so long as it allows the garment to expand while maintaining the integrity of the tissue. Lateral expansion in the rear waist area of the extensible garment is especially desirable in one aspect of the present invention. The adhesive may be applied with a discontinuity in the lateral or longitudinal direction, or both, of the tissue web. In other aspects of the invention, the adhesive can be applied as stripes or generally contiguously across the plane of the liner with a selected adhesive void area therein, and may even cover a portion of the pleats so long as some expansion is allowed.

### BRIEF DESCRIPTION OF THE DRAWINGS

The accompanying drawings are presented as an aid to explanation and understanding of various aspects of the present invention only and are not to be taken as limiting the present invention. The drawings are not necessarily to scale, nor should they be taken as photographically accurate depictions of real objects unless otherwise stated.

FIG. 1 illustrates a garment according to the present invention, in this case an exemplary diaper.

FIG. 2 illustrates components of the absorbent core in relation to the top sheet of a diaper.

FIG. 3 illustrates the unfolding of pleats on the barrier layer to allow for its expansion without tearing.

FIGS. 4-7 illustrate adhesive patterns applicable to a folded barrier layer for the absorbent core.

FIG. 8 illustrates an adhesive pattern applicable to an alternative unfolded barrier layer for the absorbent core.

FIG. 9 schematically illustrates a method for preparing a joined top sheet and absorbent core according to the present invention.

### DEFINITIONS

“Adhered” refers to the joining, adhering, connecting, attaching, or the like, of two elements. Two elements will be considered to be adhered together when they are joined directly to one another or indirectly to one another, such as when each is directly bonded to intermediate elements.

### DETAILED DESCRIPTION

The various aspects and embodiments of the invention will be described in the context of disposable absorbent articles, and more particularly referred to, without limitation and by way of illustration only, as a disposable diaper. It is, however, readily apparent that the present invention could also be employed to produce other products or garments, such as feminine care articles, various incontinence garments, medical garments and any other disposable garments. Typically, the disposable garments are intended for limited use and are not intended to be laundered or otherwise cleaned for reuse. A disposable diaper, for example, is discarded after it has become soiled by the wearer.

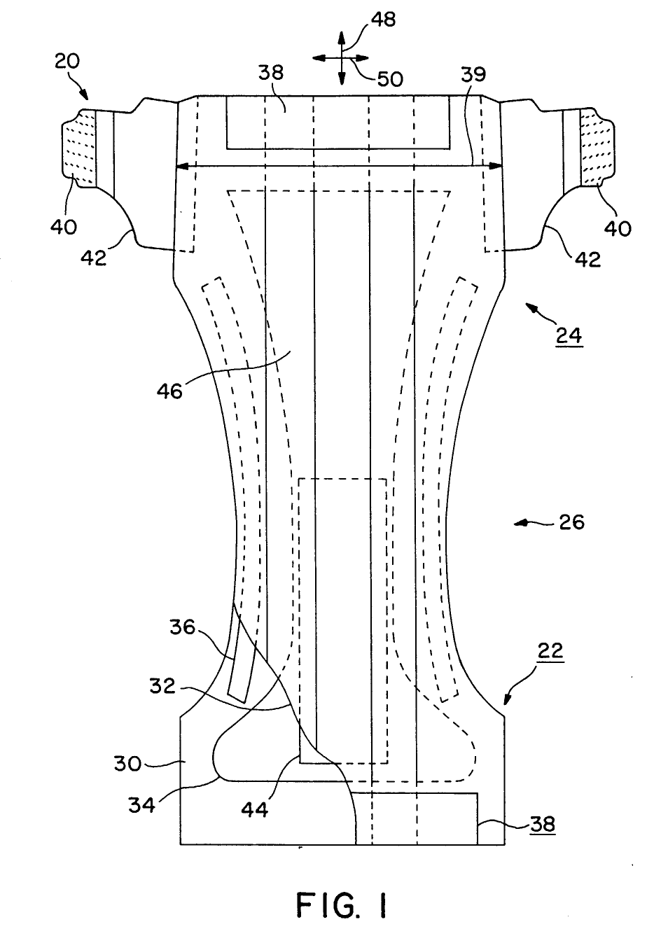

FIG. 1 is a representative plan view of an absorbent article, such as disposable diaper 20, in its flat-out, or unfolded state. Portions of the structure are partially cut away to more clearly show the interior construction of diaper 20. The surface of the diaper 20 which contacts the wearer is facing the viewer.

With reference to FIG. 1, the disposable diaper 20 generally defines a front waist section 22, a rear waist section 24, and an intermediate section 26 which interconnects the front and rear waist sections. The front and rear waist sections 22 and 24 include the general portions of the diaper which are constructed to extend substantially over the wearer's front and rear abdominal regions, respectively, during use. The intermediate section 26 of the diaper includes the general portion of the diaper that is constructed to extend through the wearer's crotch region between the legs. Thus, the intermediate section 26 is an area where repeated liquid surges typically occur in the diaper.

The diaper 20 includes, without limitation, an outer cover, or back sheet 30, a liquid permeable bodyside liner, or top sheet, 32 positioned in facing relation with the back sheet 30, and an absorbent core, or body, being the primary liquid retention structure, 34, such as an absorbent pad, which is located between the back sheet 30 and the top sheet 32. The back sheet 30 defines a length, or longitudinal direction 48, and a width, or lateral direction 50 which, in the illustrated embodiment, coincide with the length and width of the diaper 20. The liquid retention structure 34 generally has a length and width that are less than the length and width of the back sheet 30, respectively. Thus, marginal portions of the diaper 20, such as marginal sections of the back sheet 30, may extend past the terminal edges of the liquid retention structure 34. In the illustrated embodiments, for example, the back sheet 30 extends outwardly beyond the terminal marginal edges of the liquid retention structure 34 to form side margins and end margins of the diaper 20. The top sheet 32 is generally coextensive with the back sheet 30 but may optionally cover an area which is larger or smaller than the area of the back sheet 30, as desired.

The diaper 20 may include leg elastics 36 which are constructed to operably tension the side margins of the diaper 20 to provide elasticized leg bands which can closely fit around the legs of the wearer to reduce leakage and provide improved comfort and appearance. Waist elastics 38 are employed to elasticize the end margins of the diaper 20 to provide elasticized waistbands. The waist elastics 38 are configured to provide a resilient, comfortably close fit around the waist of the wearer. An exemplary zone of expansion 39 is indicated by an arrow for the diaper 20 as generally encompassing the rear waist area 24. A zone of expansion 39 will be any area of the diaper 20 made to be extensible to increase fit and comfort of some embodiments of the diaper 20. The person having ordinary skill in the art will appreciate that other areas, such as the front waist section 22, or the entire area of the diaper 20 such as covered by top sheet 32, may be made extensible.

In the illustrated embodiment, the diaper 20 includes a pair of side panels 42 to which fasteners 40, indicated as the hook portion of a hook and loop fastener, are attached. Generally, the side panels 42 are attached to the side edges of the diaper 20 in one of the waist sections 22, 24 and extend laterally outward therefrom. The side panels 42 may be elasticized or otherwise rendered elastomeric. For example, the side panels 42, or indeed, any precursor component webs of the garment, may be an elastomeric material such as a neck-bonded laminate (NBL) or stretch-bonded laminate (SBL) material. Methods of making such materials are well known to those skilled in the art and are described in U.S. Pat. No. 4,663,220 issued May 5, 1987 to Wisneski et al., U.S. Pat. No. 5,226,992 issued Jul. 13, 1993 to Morman, and European Patent Application No. EP 0 217 032 published on Apr. 8, 1987 in the names of Taylor et al. Examples of absorbent articles that include elasticized side panels and selectively configured fastener tabs are described in PCT Patent Application No. WO 95/16425 published Jun. 22, 1995 to Roessler; U.S. Pat. No. 5,399,219 issued Mar. 21, 1995 to Roessler et al.; U.S. Pat. No. 5,540,796 to Fries; and U.S. Pat. No. 5,595,618 to Fries and U.S. Pat. No. 5,496,298 to Kuepper et al.

The diaper 20 may also include a surge management layer 44, located between the top sheet 32 and the liquid retention structure 34, to rapidly accept fluid exudates and distribute the fluid exudates to the liquid retention structure 34 within the diaper 20. The surge management layer 44 may be made from extensible materials also. In some instances of the present invention, the liquid retention structure 34 may be directly adhered to the overlaying surge management layer 44 rather than the top sheet 32. It will be understood that discussion or claims herein of adhering the liquid retention structure 34 to the top sheet 32 will encompass the cases where the liquid retention structure 34 is adhered to the surge management layer 44, or to the top sheet 32, or both. The diaper 20 may further include a ventilation layer (not illustrated) located between the liquid retention structure 34 and the back sheet 30 to insulate the back sheet 30 from the liquid retention structure 34 to reduce the dampness of the garment at the exterior surface of the back sheet 30. Examples of suitable surge management layers 44 are described in U.S. Pat. No. 5,486,166 to Bishop and U.S. Pat. No. 5,490,846 to Ellis; U.S. Pat. No. 5,364,382 to Latimer et al.; and U.S. Pat. No. 5,429,629 to Latimer et al.

As representatively illustrated in FIG. 1, the disposable diaper 20 may also include a pair of containment flaps 46 which are configured to provide a barrier to the lateral flow of body exudates. The containment flaps 46 may be located along the laterally opposed side edges of the diaper 20 adjacent the side edges of the liquid retention structure 34. Each containment flap 46 typically defines an unattached edge which is configured to maintain an upright, perpendicular configuration in at least the intermediate section 26 of the diaper 20 to form a seal against the wearer's body.

The back sheet 30 and the top sheet 32 are most desirably constructed to be extensible in the lateral direction 50 in at least the rear waist section 24 so as to provide a better fit, as previously mentioned.

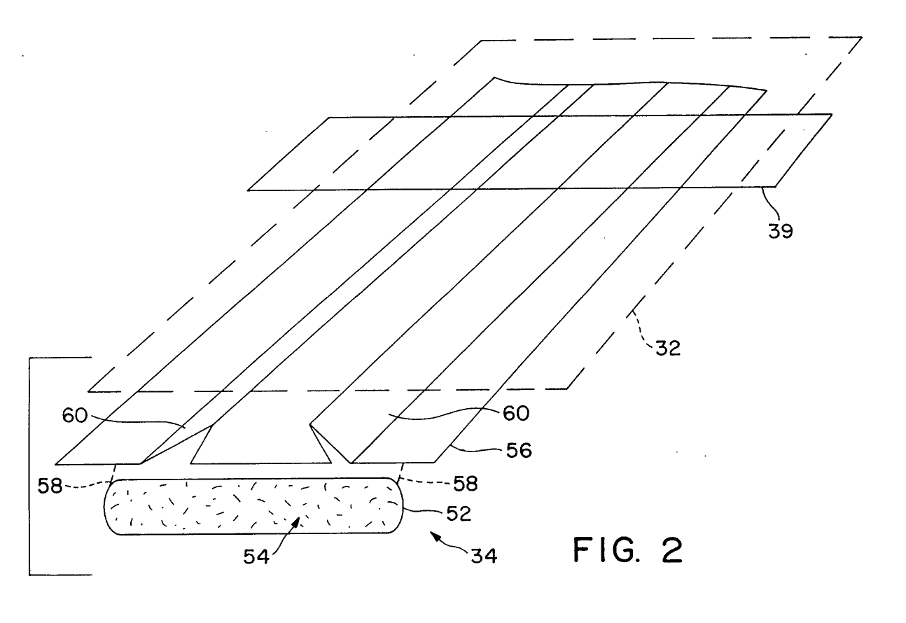

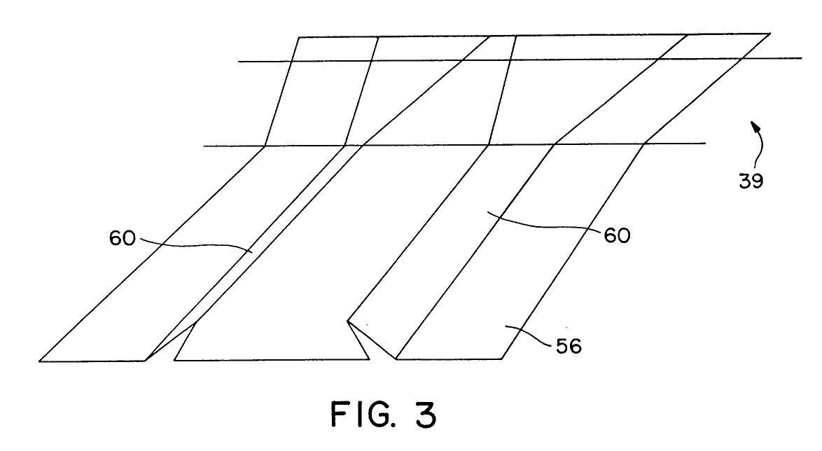

Referring also to FIG. 2, a perspective view shows the top sheet 32 (in phantom) in position over the absorbent core 34 including a fluff pad, or web, 52 carrying superabsorbent particles (SAP) 54 and the pleated tissue barrier layer 56 shown as separated from the fluff pad 52 for ease of understanding, although it will be appreciated that the barrier layer 56 is attached to the fluff pad 52 in actual construction of the diaper (20, FIG. 1) before attachment to the top sheet 32, as indicated by dotted lines 58, and further explained below. The pleats 60 of the barrier tissue 56 expand or unfold, as shown in FIG. 3, to enable the barrier tissue 56 to expand in the zone of lateral expansion 39 without tearing to accommodate stretching of the diaper 20 (FIG. 1) when practiced according to aspects of the present invention. The absorbent core 34 and top sheet 32 layers are illustrated in FIG. 2 as untrimmed or unshaped for ease of understanding, but it will be appreciated by the person having ordinary skill in the art that trimming of the layers may take place in accordance with known methods of the art. The back sheet 30 is not illustrated in FIG. 2.

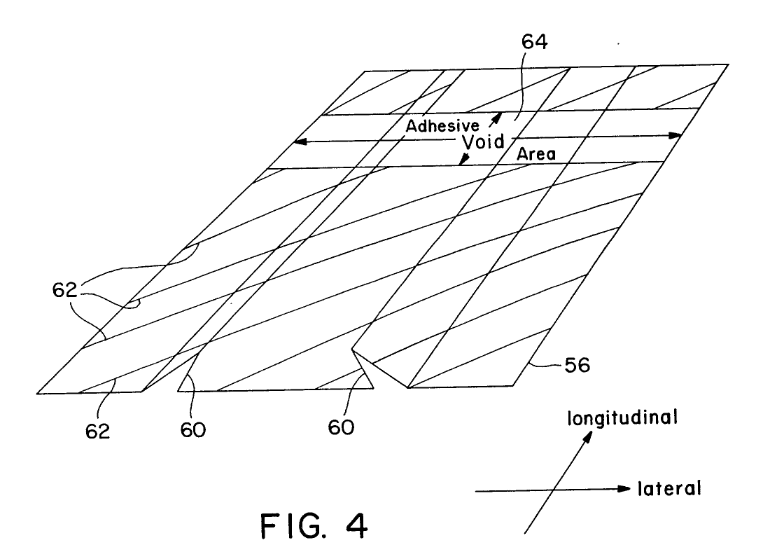

Referencing FIG. 4, a pleated barrier tissue 56 is shown with an adhesive 62 applied as a layer to that surface of the barrier tissue 56 meant to contact and adhere the absorbent core 34 to the top sheet 32 (FIG. 2). The adhesive is applied in a substantially contiguous manner across the surface of the tissue 56 except in an adhesive void area 64 which generally corresponds to the area of the zone of expansion 39 for the diaper 20 (FIG. 1). By placing the adhesive void area 64 over the pleats 60 in the zone of expansion 39, the pleats 60 in that area are allowed to unfold when the diaper 20 laterally expands, thus preventing the barrier tissue 56 from tearing, thereby maintaining the integrity of the diaper construction.

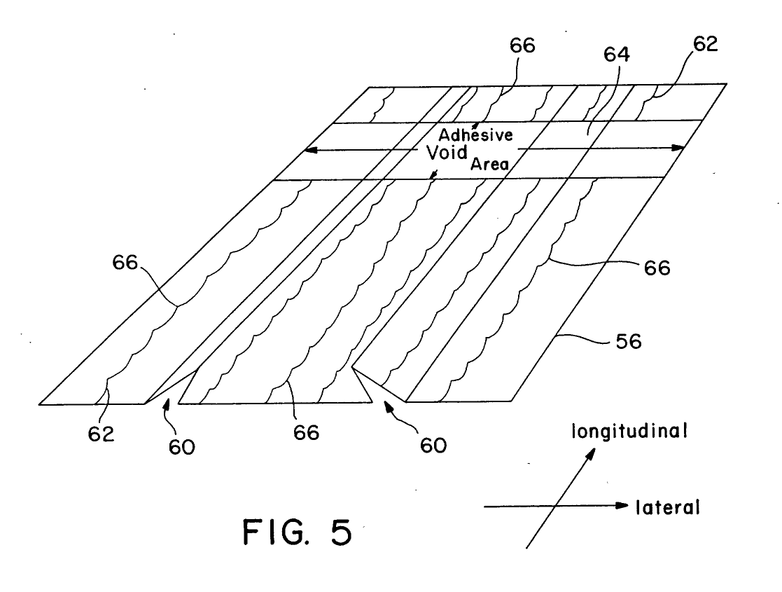

Referencing FIG. 5, the pleated barrier tissue 56 is shown with an adhesive 62 applied to that surface of the barrier tissue 56 meant to contact and adhere the absorbent core 34 to the top sheet 32 (FIG. 2). The adhesive 62 is applied in longitudinally extending stripes 66 across the surface of the tissue 56 except in the adhesive void area 64 which generally corresponds to the area of the zone of expansion 39 for the diaper 20 (FIG. 1). The stripes may be patterned in such a manner as repeating swirls, a sine wave, or the like, if necessary or desired. By placing the adhesive void area 64 over the pleats 60 in the zone of expansion 39, the pleats in that area are allowed to unfold when the diaper 20 laterally expands, thus preventing the barrier tissue 56 from tearing and maintaining the integrity of the diaper construction. It will be appreciated that the longitudinally extending stripes 66 may be applied over the pleats 60 in all areas except that of the adhesive void area 64 according to this aspect of the invention.

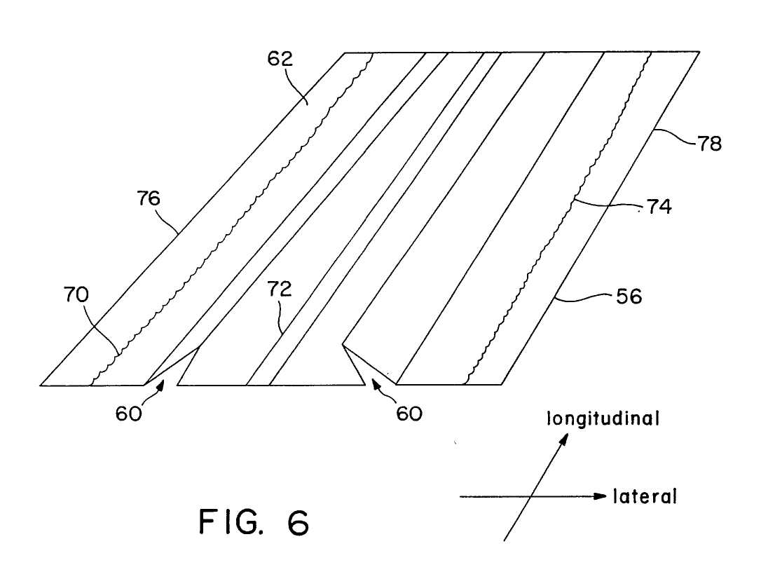

Referencing FIG. 6, the pleated barrier tissue 56 is shown with an adhesive 62 applied to that surface of the barrier tissue 56 meant to contact and adhere the absorbent core 34 to the top sheet 32 (FIG. 2). The adhesive 62 is applied in three longitudinally extending stripes 70, 72, and 74 along the entire length of the surface of the tissue 56. The outer stripes 70, 74 are placed towards the longitudinal edges 76, 78 of the barrier tissue 56 and outside of the pleats 60. The center stripe 72 is placed towards the longitudinal midline of the barrier tissue 56 and between the pleats 60. Thus the entire length of the barrier tissue 56 is free to expand laterally with an adhered top sheet 32 (FIG. 2) without interference to the pleats 60. No break in the application of the longitudinally extending stripes 70, 72, 74 is necessary to create an adhesive void area, further making adhesive application easy. While the lines of outer stripes 70, 74 show the application of the adhesive 62 as swirled, and center stripe 72 is shown in a thicker continual application, it will be understood that they may be applied in any such configuration as is necessary to accomplish the present purpose.

For comparison, in confidential field trials, 35.4% of diapers with a single stripe of adhesive down the longitudinal center line were reported having a gel on skin occurrence. Diapers utilizing the same nonwoven liner material (0.6 osy necked spunbond) and the same 21.1 gsm tissue material (e.g., American Tissue 12.5 pound per ream white tissue from American Tissue, Inc. of Neenah, Wis.), but in the adhesive pattern of the embodiment of FIG. 6, were reported with a significant reduction of gel on skin occurrence down to a range between 13.9% to 20.1%. Without being bound by theory, it is believed that the additional adhesive allowed a reinforcement of the tissue preventing more tearing. Thus, the addition of more adhesive, e.g. as shown in FIGS. 4, 5, 7 and 8, while allowing sufficient extensibility of the liner, may produce additional reduction of reported gel on skin occurrence.

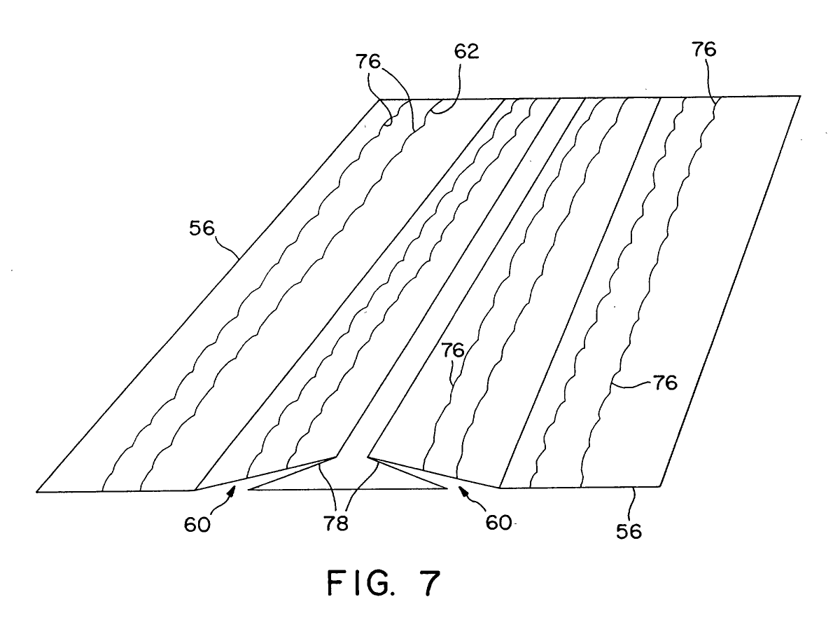

Referencing FIG. 7, the pleated barrier tissue 56 is shown with an adhesive 62 applied to that surface of the barrier tissue 56 meant to contact and adhere the absorbent core 34 to the top sheet 32 (FIG. 2). The adhesive 62 is applied in multiple longitudinally extending stripes, collectively 76, along the entire length of the surface of the tissue 56. In this aspect of the invention, the pleats 60 may be made fairly wide and the stripes 76 are placed across the entire surface of the tissue 56 except for that apex portion 78 of the pleats 60 which is required to unfold to maintain integrity of the barrier tissue 56 during lateral expansion of the diaper 20. Any gap that may exist between the apex portions is also left without adhesive. This alternative again allows the entire length of the barrier tissue 56 to laterally expand with the adhered top sheet without interference to the functional portion of the pleats 60. Again, no break in the longitudinal application of the longitudinally extending stripes 66 is necessary to create an adhesive void area, further making adhesive application easy, and in some instances, more adhesive may be applied than in the embodiment of FIG. 6, if desired.

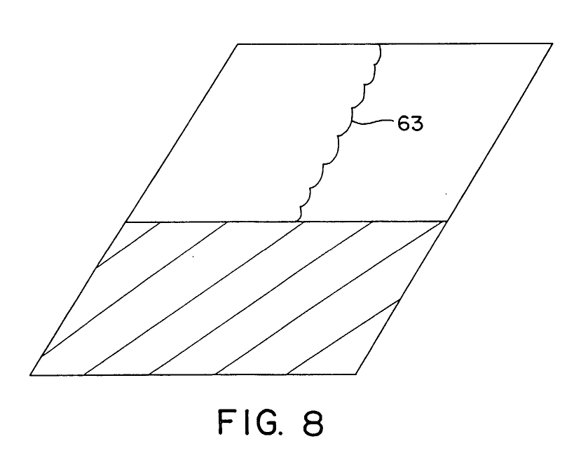

Referencing FIG. 8, an unplcated embodiment of the barrier tissue 56 is shown with an adhesive 62 applied as a layer to that surface of the barrier tissue 56 meant to contact the front waist section 22 of the top sheet 32 (FIG. 2). The adhesive 62 is applied in a substantially contiguous manner across the surface of the tissue 56 except in an adhesive void area 64 which generally corresponds to the entire rear waist section 24 area for the diaper 20 (FIG. 1). By leaving the rear waist section void of adhesive, the absorbent pad is allowed to moved unrestrictedly beneath the inner liner when the diaper 20 laterally expands, thus preventing the barrier tissue 56 from tearing, thereby maintaining the integrity of the diaper construction. Alternatively, a single line of adhesive 63 may be placed along the longitudinal center line instead of leaving a complete void of adhesive, as is true for the embodiments of FIGS. 4 and 5 also.

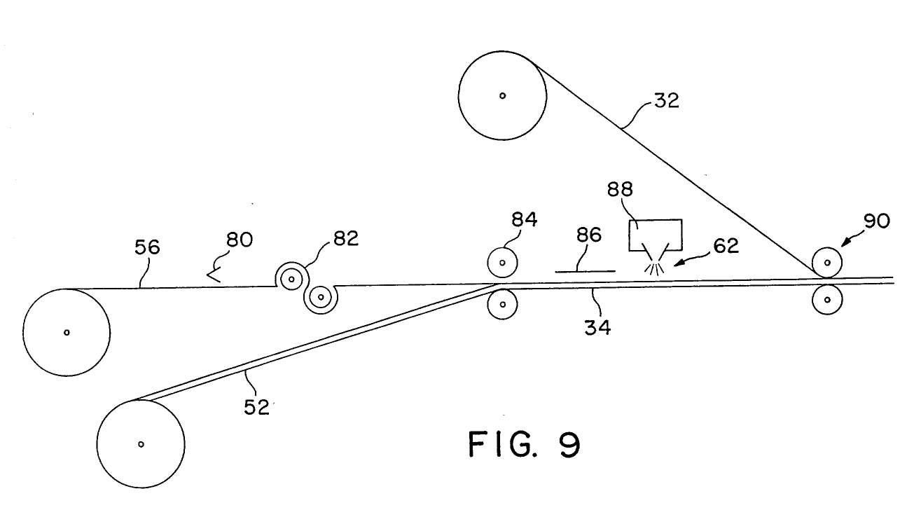

Referencing FIG. 9, a method of constructing the joined absorbent core 34 and top sheet 32 is schematically illustrated. The pleats (not shown) are formed by a pleater 80 in known fashion just before the barrier tissue 56 joins the fluff pad 52 with the superabsorbent (54 not shown) therein. An idler 82 immediately downstream of the pleater 80 sets the creases that form the apex and base of each pleat. The greater the wrap angle on the idler 82, and the smaller the diameter of idler 82, the better the pleats are creased into the tissue. A deadbar or a driven roll may also function acceptably in place of the idler 82. A debulker 84 compresses the pleats of the barrier tissue 56 down to the fluff pad 52, creating a light hydrogen bond between the multiple layers of the absorbent core 34. The surface of one or both rolls of the debulker 84 can be contoured such that the gap between them varies either laterally, longitudinally, or both, so that selected regions of the pleats receive more pressure than others. Greater pressure can help maintain pleat retention through the diaper construction process. Care should be taken that the pressure is not so great that it cuts the tissue. The light bond is then relied upon to keep the pleats in the closed position throughout the remainder of the diaper construction process. Adhesive 62 is applied to the barrier tissue side of the joined absorbent core 34 in the exemplary embodiment by one or more spray nozzles 88. Placement and timed control of the nozzles may be utilized for intermittent or discontinuous application of the adhesive in either or both of the lateral and longitudinal directions of the garment. Other suitable means may be employed, such as printing techniques or the like, to achieve the desired pattern of application for the adhesive in keeping with the teachings herein. The absorbent core 34 is then brought into contact with the top sheet layer 32, as at nip rollers 90.

Barrier tissue pleats were formed at 10 mm and 13 mm high during experiments with the present invention. The 10 mm pleats were subject to being pulled open as they passed water cutter trim takeaway vacuum chutes during forming of the tissue. Pleats also opened up at the infeed to the fluff cutoff during absorbent core shaping. Therefore, the person having ordinary skill in the art will understand that a hold-down ski, or mat, 86 may be placed at any of various points of the process where needed to increase the percentage of pleats remaining closed during processing. Tall tissue pleats located adequately inboard of the absorbent core edges were found to be easier to keep closed through the diaper construction process than were shorter pleats located nearer the absorbent core edges.

While the invention has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

We claim:

1. An absorbent article comprising:
a) an extensible top sheet for facing the body of a wearer and a back sheet serving as an outer cover for the article;
b) an absorbent core adhered to at least one of the top sheet and the back sheet, the absorbent core having a tissue web acting as a boundary layer between at least one of the top sheet and the back sheet and the remainder of the absorbent core; and
c) wherein the tissue web of the absorbent core has an adhesive discontinuously applied thereto for adhering to at least one of the top sheet and the back sheet.

2. The absorbent article according to claim 1 wherein the tissue web of the absorbent core is pleated.

3. The absorbent article according to claim 1 wherein the absorbent core contains superabsorbent particles.

4. The absorbent article according to claim 1 wherein the adhesive is discontinuous in a lateral direction of the tissue web.

5. The absorbent article according to claim 4 wherein the adhesive is applied in lines extending in a longitudinal direction of the tissue web.

6. The absorbent article according to claim 1 wherein the adhesive is discontinuous in a longitudinal direction of the tissue web.

7. The absorbent article according to claim 6 wherein the adhesive is otherwise applied contiguously over the surface of the tissue web.

8. The absorbent article according to claim 6 wherein the adhesive is applied in lines extending in the longitudinal direction of the tissue web.

9. The absorbent article according to claim 1 wherein the adhesive is discontinuous in a longitudinal direction and is discontinuous in a lateral direction of the tissue web.

10. The absorbent article according to claim 9 wherein the adhesive is applied in lines extending in a longitudinal direction of the tissue web.

11. The absorbent article according to claim 2 wherein the discontinuity of adhesive leaves the pleated tissue web free to expand.

12. An absorbent article having:
a) an extensible top sheet for facing the body of a wearer;
b) a back sheet for an exterior layer facing away from the body of the wearer;
c) an absorbent core located between the top sheet and the back sheet, the absorbent core having a tissue web acting as a boundary layer between the top sheet and the remainder of the absorbent core; and
d) wherein the tissue web of the absorbent core is pleated and has an adhesive discontinuously applied thereto for attachment to the top sheet.

13. The absorbent article according to claim 12 wherein the absorbent core contains superabsorbent particles.

14. The absorbent article according to claim 12 wherein the adhesive is discontinuous in a lateral direction of the tissue web.

15. The absorbent article according to claim 14 wherein the adhesive is applied in lines extending in a longitudinal direction of the tissue web.

16. The absorbent article according to claim 12 wherein the adhesive is discontinuous in a longitudinal direction of the tissue web.

17. The absorbent article according to claim 16 wherein the adhesive is otherwise applied contiguously over the surface of the tissue web.

18. The absorbent article according to claim 16 wherein the adhesive is applied in lines extending in the longitudinal direction of the tissue web.

19. The absorbent article according to claim 12 wherein the adhesive is discontinuous in a longitudinal direction and is discontinuous in a lateral direction of the tissue web.

20. The absorbent article according to claim 19 wherein the adhesive is applied in lines extending in the longitudinal direction of the tissue.

21. The absorbent article according to claim 12 wherein the discontinuity of adhesive leaves the pleated tissue web free to expand.

22. The absorbent article according to claim 12 wherein the absorbent article is an incontinence garment.

23. The absorbent article according to claim 22 wherein the incontinence garment is extensible in a lateral direction of the incontinence garment.

24. The absorbent article according to claim 22 wherein the discontinuity of adhesive is positioned in a zone of lateral expansion of the incontinence garment.

25. A method of making an absorbent article comprising:
a) supplying an extensible top sheet for facing the body of a wearer;
b) supplying an extensible back sheet for an exterior layer facing away from the body of the wearer;
c) providing an absorbent web having superabsorbent particles therein;
d) joining the absorbent web with a pleated tissue web which acts as a boundary layer for retaining the superabsorbent particles within the absorbent web;
e) supplying the joined absorbent web and pleated tissue web as an absorbent core for a primary liquid retention layer of the absorbent article;
f) discontinuously applying an adhesive to the pleated tissue web for attachment to at least one of the top sheet and the back sheet;
g) joining the pleated tissue web with adhesive thereon to at least one of the top sheet or the back sheet; and
h) locating the absorbent core between the top sheet and the back sheet.

26. The method according to claim 25 further comprising: applying adhesive in longitudinal stripes which do not touch the pleats whereby the adhesive has a discontinuity in a lateral direction of the tissue web.

27. The absorbent article according to claim 25 wherein the adhesive has a discontinuity in the longitudinal direction of the tissue web.

28. The absorbent article according to claim 27 wherein the adhesive is otherwise applied contiguously over the surface of the tissue web.

29. The absorbent article according to claim 27 wherein the adhesive is applied in lines extending in a longitudinal direction of the tissue web.

30. The absorbent article according to claim 25 wherein the adhesive has a discontinuity in a longitudinal direction and a discontinuity in a lateral direction of the tissue web.

31. The method according to claim 25 further comprising: applying adhesive in longitudinal stripes which touch a portion of the pleats, whereby the adhesive has a discontinuity in a lateral direction of the tissue web.

32. An absorbent article having:
a) a top sheet for facing the body of a wearer and a back sheet for an exterior layer facing away from the body of the wearer, at least one of the top sheet and back sheet being extensible between a first unextended position and a second extended position;
b) an absorbent core located between the top sheet and the back sheet, the absorbent core having a tissue web acting as a boundary layer between the top sheet and the remainder of the absorbent core; and
d) wherein the tissue web of the absorbent core has an adhesive discontinuously applied thereto and is attached thereby to at least one of the top sheet and the bottom sheet; and
e) whereby, as the at least one of the top sheet and back sheet is extended between the first unextended position and the second extended position the tissue web remains as a boundary layer.
